(19) United States
(12) Patent Application Publication
(10) Pub. No.: US 2005/0171495 A1
(43) Pub. Date: Aug. 4, 2005

(54) Waste collection unit with manifold interface assembly

(76) Inventors: Timothy William Austin, Scotts, MI (US); Stephen J. Reasoner, Kalamazoo, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/046,289

(22) Filed: Jan. 28, 2005

Related U.S. Application Data
(60) Provisional application No. 60/540,267, filed on Jan. 29, 2004.

(51) Int. Cl. A61M 1/00

(52) U.S. Cl. 604/317

(57) ABSTRACT

A waste collection unit is provided for collecting medical waste from a surgical site. The waste collection unit comprises a canister for collecting the medical waste. A disposable manifold is coupled to the canister to filter solid and semi-solid debris from the medical waste prior to the medical waste entering the canister. A manifold interface assembly of the waste collection unit provides a valve assembly having a valve body and flapper door to prevent the escape of the medical waste from the canister when the manifold is removed from the canister. The manifold interface assembly is configured such that the flapper door is engaged by the manifold upon insertion of the manifold into the canister. In particular, the manifold includes a stem that is sized for inserting into an inlet port of the canister and the stem has a length sufficiently long to engage and open the flapper door upon insertion.

### CROSS-REFERENCE TO RELATED APPLICATIONS

This application claims the benefit of U.S. provisional patent application Ser. No. 60/540,267, filed on Jan. 29, 2004, the advantages and disclosure of which are herein incorporated by reference.

### FIELD OF THE INVENTION

The present invention relates generally to waste collection units for use in collecting medical waste such as bodily fluids. More specifically, the present invention relates to an improved manifold interface assembly for use with such waste collection units.

### BACKGROUND OF THE INVENTION

Waste collection units are well known for use in surgical environments to collect medical waste such as bodily fluids during a surgical procedure. Examples of waste collection units can be found in United States Pat. Nos. 5,997,733; 6,180,000; and 6,222,283. For instance, U.S. Pat. No. 5,997,733 discloses a waste liquid and smoke disposal system which combines the functions of a smoke extraction system and a waste collection unit, typically in, but not limited to, a surgical environment. The smoke extraction system and the waste collection unit are connected to supply the medical waste collected thereby to a waste treatment (e.g. decontamination and/or sterilization) and disposal system. These three systems are all combined into an integrated system wherein the treated medical waste can be safely returned to the ambient. In one embodiment, the integrated system is provided as a cart-mounted apparatus to provide mobility. With this type of system, a surgical team can quickly, easily, and efficiently maintain the integrity of a surgical site with a minimum of operating components.

Disposable manifolds are used to facilitate the collection of the medical waste into the waste collection unit. Typically, the manifolds include at least one filter to remove solid or semi-solid material such as bone chips, flesh, blood clots or the like from the medical waste generated by the surgical procedure or operation. The manifolds are disposed of between patients, or when the manifold is spent, i.e., filled with solid and semi-solid materials. An example of a disposable manifold for use in waste collection units is described in U.S. Pat. No. 6,331,246.

Currently, the waste collection unit includes a waste container that contains the medical waste after being filtered by the manifold. The waste container includes an inlet port and the manifold includes a stem that is inserted into the inlet port to couple the manifold to the waste container. A vacuum is applied within the waste container during use to draw the medical waste into the manifold from the surgical site and through the filter to the waste container, while the manifold filters out the solid and semi-solid waste materials. As a result, the waste container is typically filled with liquid waste. When manifolds are removed, such as to insert a new manifold, or when transporting the waste collection unit for disposal and/or cleaning, the inlet port remains open, which increases the likelihood that the liquid waste will spill out of the waste container through the inlet port. As a result, there is a need in the art to close the inlet port when changing manifolds or transporting the waste collection unit for disposal of the medical waste contained therein.

### BRIEF SUMMARY OF THE INVENTION AND ADVANTAGES

The present invention provides a waste collection unit for handling medical waste. The waste collection unit includes a waste container having an inlet port. A manifold is provided for filtering the medical waste prior to the medical waste entering the waste container. The manifold includes at least one filter to serve this purpose. The manifold also includes a stem sized for inserting into the inlet port to couple the manifold to the waste container. A manifold interface assembly is provided for interacting with the manifold. The manifold interface assembly includes a valve. The valve includes a valve body, a flapper door mounted to the valve body to move between open and closed positions relative to the valve body, and a biasing device to normally bias the flapper door into the closed position to close the inlet port. The stem of the manifold has a length sufficiently long to engage the flapper door and force the flapper door into the open position upon inserting the stem into the inlet port.

This configuration has the advantage of closing off the inlet port of the waste container when manifolds are being replaced or the waste collection unit is being transported for disposal and/or cleaning. Otherwise, without this manifold interface assembly, the inlet port would remain open when the manifold is removed thus allowing liquid medical waste captured in the waste container to potentially spill out of the waste container through the inlet port.

### BRIEF DESCRIPTION OF THE SEVERAL VIEWS OF THE DRAWINGS

Advantages of the present invention will be readily appreciated as the same becomes better understood by reference to the following detailed description when considered in connection with the accompanying drawings wherein:

FIG. 1 is a perspective view of a waste collection unit of the present invention;

FIG. 2 is an exploded perspective view of a manifold interface assembly and manifold of the waste collection unit;

FIG. 3 is a top view of a valve body;

FIG. 4 is a cross-sectional view of the valve body taken along the line 4-4 in FIG. 3;

FIG. 5 is a top perspective view of the flapper door;

FIG. 6 is a cross-sectional view of the flapper door taken along the line 6-6 in FIG. 5;

FIG. 7 is a blown-up cross-sectional view of a retainer and seal of the manifold interface assembly from FIG. 3;

FIG. 8 is a cross-sectional view of the manifold interface assembly illustrating the manifold being partially inserted into an inlet port with a flapper door in a normally closed position;

FIG. 9 is a cross-sectional view of the manifold interface assembly illustrating the manifold being fully inserted into the inlet port to engage the flapper door and move the flapper door to an open position; and

FIG. 10 is a cross-sectional view of an alternative manifold interface assembly.

### DETAILED DESCRIPTION OF THE INVENTION

Referring to the Figures, wherein like numerals indicate like or corresponding parts throughout the several views, a waste collection unit 10 for handling medical waste is generally shown at 10. The waste collection unit 10 is part of an integrated medical waste collection, treatment, and disposal system that provides for overall management of medical waste in surgical environments. The integrated system is best described in United States Pat. Nos. 5,997,733; 6,180,000; and 6,222,283, all of which are herein incorporated by reference. In general, the integrated system combines the functions of a smoke extraction system and a liquid waste collection system, typically in, but not limited to, a surgical environment with a waste treatment (e.g. decontamination and/or sterilization) and disposal system. In one embodiment, the integrated system is provided as a cart-mounted apparatus to provide mobility. An example of such an integrated system is the NEPTUNE™ Waste Management System commercially available from Stryker Instruments of Kalamazoo, Mich., the current assignee.

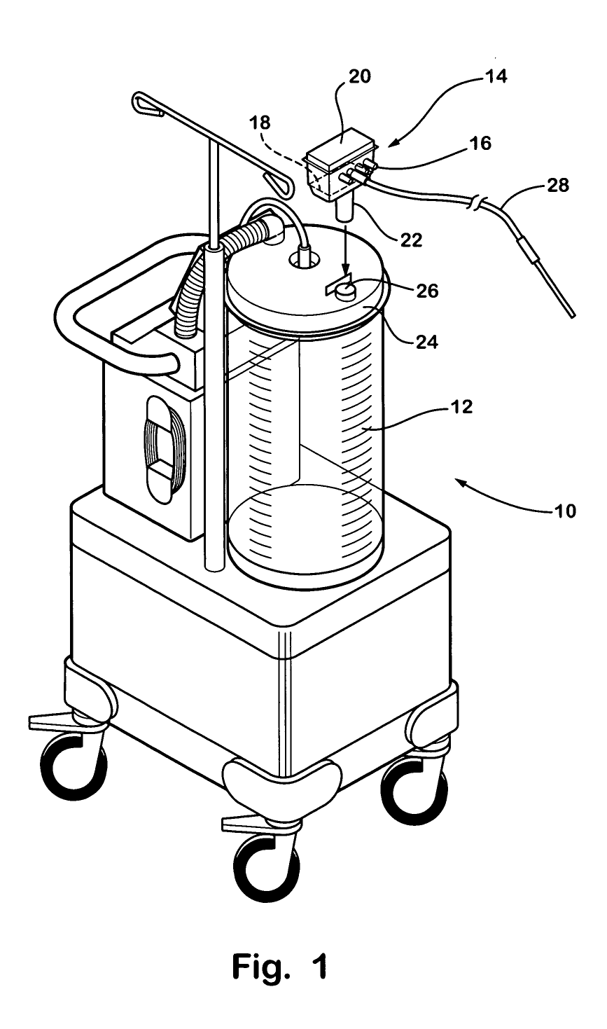

Referring to FIG. 1, the present invention provides an improved interface between a waste container 12, e.g., canister 12, of the waste collection unit 10 and disposable manifolds 14 used with the waste collection unit 10. Preferably, the manifolds 14 include at least one inlet 16 for receiving the medical waste from a surgical site and at least one filter 18 (typically multiple filters of varying porosity) for filtering the medical waste prior to the medical waste entering the canister 12. Examples of such a manifold 14 are shown in U.S. Pat. No. 6,331,246 and in United States Patent Application Publication No. 2004/0016691, both of which are herein incorporated by reference. Similar manifolds are commercially available from Stryker Instruments of Kalamazoo, Mich., the current assignee.

Each of the manifolds 14 includes a body 20 and a stem 22 extending downwardly from the body 20 for inserting into the canister 12. The canister 12 includes a cap 24 and the cap 24 defines an inlet port 26 for receiving the stem 22 of the manifold 14. The manifolds 14 are used for the removal of solid or semi-solid material such as bone chips, flesh, blood clots or the like from the medical waste prior to the medical waste entering the canister 12. This removal process permits the liquid or fluid carrier to be treated separately from the other debris, which is trapped by the manifold 14. During a typical surgical procedure, a vacuum is applied inside the canister 12 and suction tubes 28 are connected to the inlets 16 of the manifold 14 to draw the medical waste from the surgical site, through the suction tubes 28, and across the at least one filter 18 and down the stem 22 into the canister 12 to be collected. The manifolds 14 are disposed of between patients, or when the manifold 14 is spent, i.e., filled with solid and semi-solid materials.

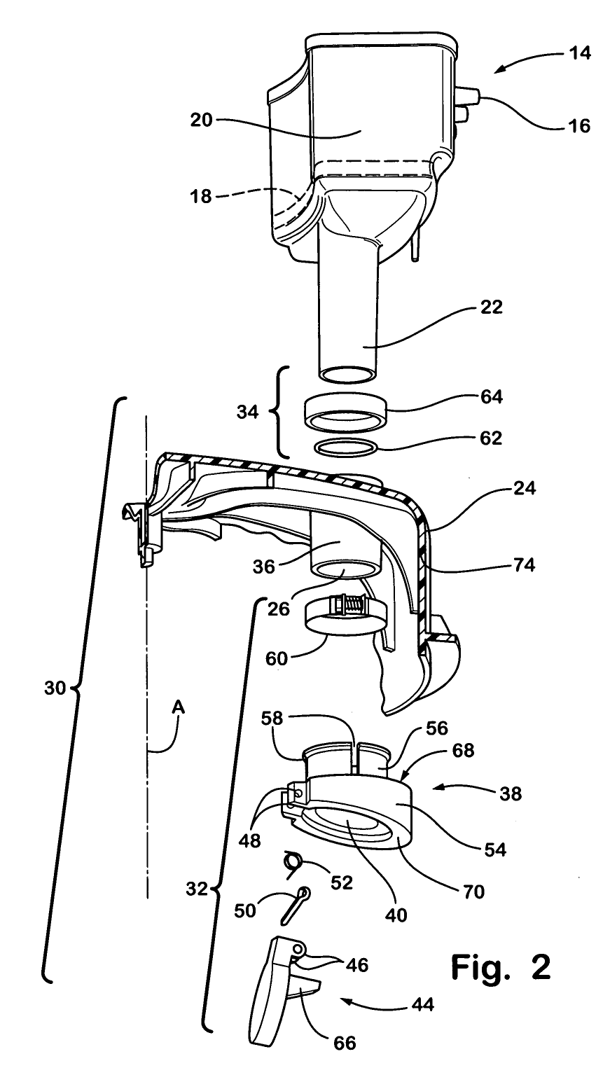

Referring to FIG. 2, a manifold interface assembly of the present invention is generally shown at 30. The manifold interface assembly 30 is divided into two separate assemblies. The first is a valve assembly generally shown at 32. The second is a retainer and seal assembly generally shown at 34. These assemblies 32, 34 are mounted to the cap 24 of the canister 12 (only a portion of the cap 24 is shown in FIG. 2 for ease of illustration). More specifically, these assemblies 32, 34 are mounted to an inlet member 36 of the cap 24. The inlet member 36 defines the inlet port 26. The inlet member 36, with the manifold interface assembly 30 mounted thereto, is adapted to receive the stem 22 of the manifold 14. In the disclosed embodiment, the stem 22 is tapered from the body 20 to a distal end of the stem 22 to facilitate inserting the stem 22 into the inlet port 26.

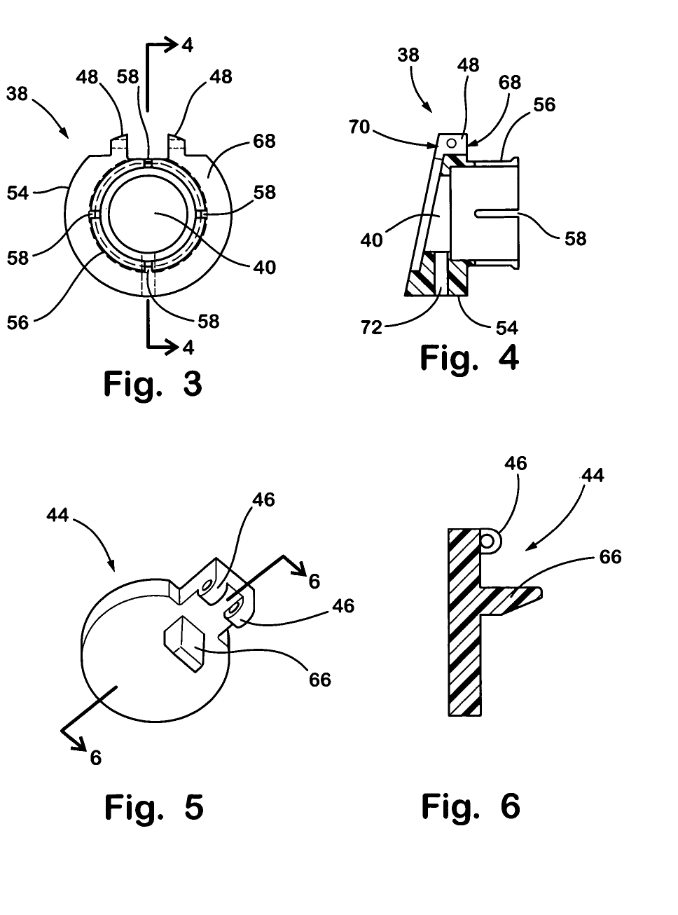

The valve assembly 32 includes a valve body 38 defining a throat 40 for receiving the stem 22 of the manifold 14. The throat 40 extends through the valve body 38 for receiving the tapered stem 22 to provide a tight seal between the stem 22 and an inner surface of the throat 40. The valve assembly 32 also includes a movable member 44 that is pivotally supported by the valve body 38. The movable member 44 is preferably in the form of a flapper door 44. The flapper door 44 includes a first pair of hinge arms 46 and the valve body 38 includes a second pair of hinge arms 48. A pivot pin 50 (here in the form of a cotter pin) engages the hinge arms to define a hinge for pivotally coupling the flapper door 44 to the valve body 38. The hinge is spring loaded in the disclosed embodiment by a biasing device 52. A torsion spring 52 is illustrated as the biasing device 52. The torsion spring 52 acts between the valve body 38 and the flapper door 44 to normally bias the flapper door 44 into a closed position to close the inlet port 26 and throat 40 of the valve body 38 when there is no manifold 14 present in the inlet port 26. Additional views of the valve body 38 and flapper door 44 are shown in FIGS. 3-6. Preferably, the valve body 38 and flapper door 44 are formed of thermoplastic material, more preferably polyvinyl chloride (PVC).

Still referring to FIG. 2, the valve body 38 includes a base 54 and a collar 56 extending upwardly from the base 54 to connect the valve body 38 to the inlet member 36. The collar 56 defines a plurality of slits 58 for providing flexibility to the collar 56 to allow the collar 56 to be clamped about the inlet member 36. The valve assembly 32 further includes a clamp 60 disposed about the collar 56 for securing the valve body 38 to the inlet member 36. The clamp 60 is preferably a conventional hose clamp 60 that can be tightened about the collar 56 to constrict the collar 56 about the inlet member 36 to secure the collar 56 thereto. As illustrated, the inlet member 36 is a tube formed integrally with the cap 24 of the canister 12. The collar 56 is positioned over the tube and the clamp 60 is tightened to mount the valve body 38 to the tube. It should be appreciated that the illustrated method of mounting the valve body 38 to the tube is by way of example only and numerous other methods could be used, for example, integrally forming it or welding it to the tube, using mating clips in the collar 56 and the tube, etc.

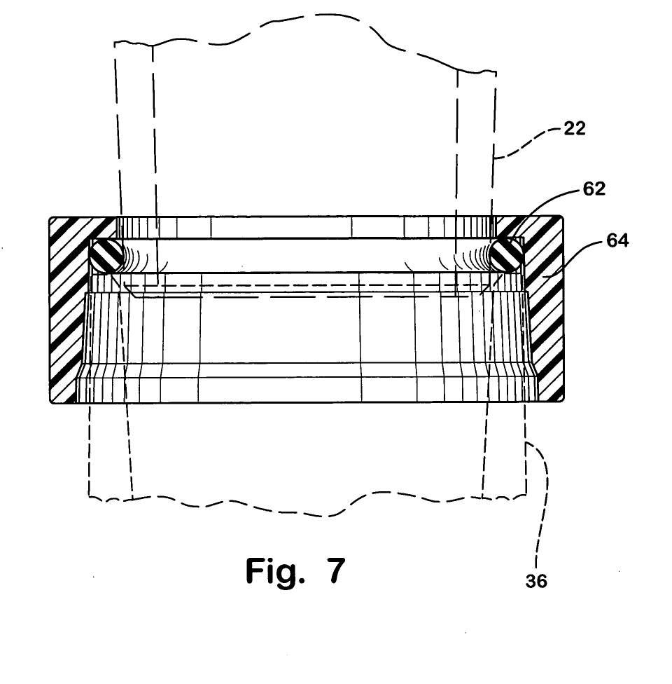

Referring to FIG. 7, the retainer and seal assembly 34 further seal the stem 22 in the inlet port 26 when the manifold 14 is coupled to the canister 12. The retainer and seal assembly 34 includes a seal 62 that sealably engages the stem 22 upon inserting the stem 22 into the inlet port 26. The retainer and seal assembly 34 further includes a retainer 64 mounted to the inlet member 36 about the inlet port 26 with the seal 62 being captured between the retainer 64 and the inlet member 36. The retainer 64 is preferably formed of a thermoplastic material, more preferably PVC. The retainer 64 is fixed to the inlet member 36 by an adhesive. Furthermore, the retainer 64 may include a stepped, inner surface to facilitate the fit between the retainer 64 and inlet members 36 of varying sizes. The seal 62 is preferably an o-ring encapsulated in a fluoropolymer. The fluoropolymer employed is preferably fluorinated ethylene propylene (FEP). The seal 62 engages the stem 22 to seal the stem 22 within the inlet port 26 to facilitate the creation of a vacuum in the canister 12, i.e., by providing a vacuum seal. The seal 62 also wipes the stem 22 when the stem 22 is removed from the canister 12 to facilitate clean removal of the manifold 14.

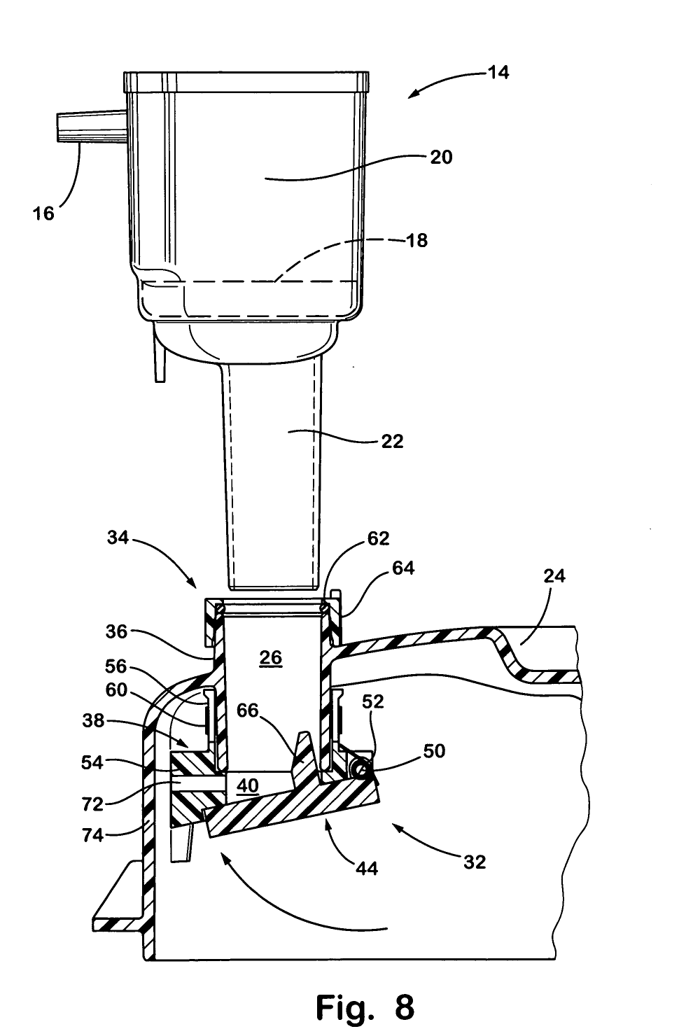

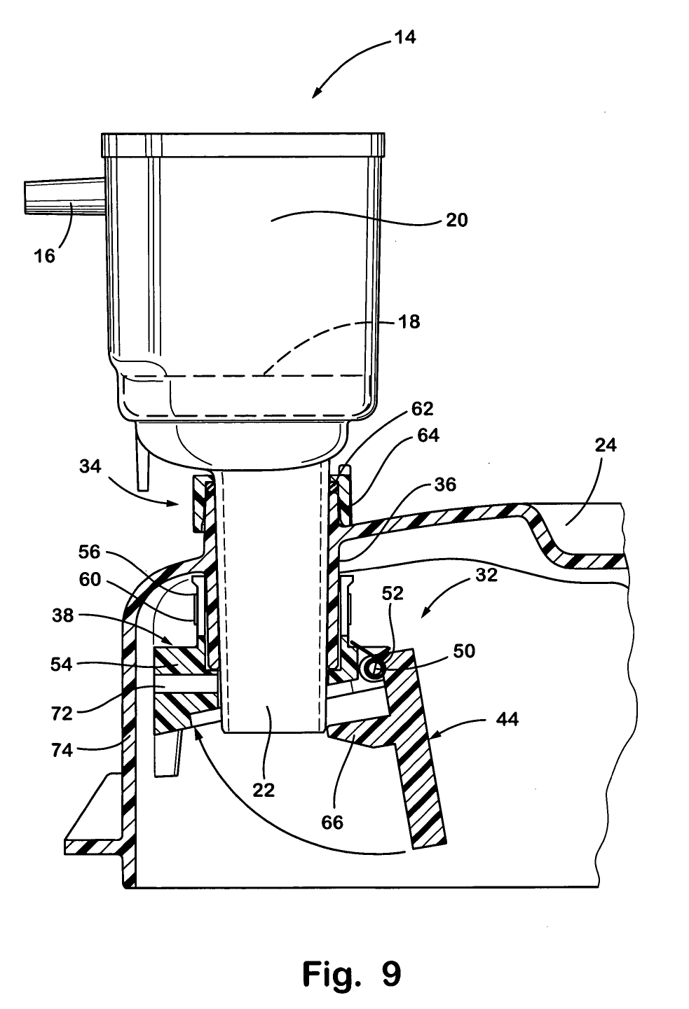

FIGS. 8 and 9 illustrate the insertion of the stem 22 of the manifold 14 into the inlet port 26. As illustrated, the flapper door 44 moves between open (FIG. 9) and closed (FIG. 8) positions relative to the valve body 38 with the flapper door 44 being normally biased into the closed position by the biasing device 52, e.g., torsion spring 52, to normally close the inlet port 26 and throat 40 of the valve body 38 when the manifold 14 is not in place. As illustrated in FIG. 9, the flapper door 44 is forced to the open position when the stem 22 of manifold 14 is inserted. The stem 22 of the manifold 14 has a length sufficiently long to engage the flapper door 44 and force the flapper door 44 into the open position upon inserting the stem 22 into the inlet port 26 so that there is no interference between the flow of fluid into the canister 12 and the flapper door 44. Thus, the stem 22 acts as an actuator of the manifold 14 to actuate the valve assembly 32 and move the flapper door 44 to the open position. The seal 62 also provides friction to hold the manifold 14 in place and counter the bias on the flapper door 44.

A finger 66 extends from the flapper door 44 and is engaged by the stem 22 when the stem 22 is inserted into the inlet port 26 and throat 40. The stem 22 of the manifold 14 engages the finger 66 to push the flapper door 44 back out of the path of fluid flowing through the stem 22. In this way, the fluid passing through the manifold 14 entirely misses the flapper door 44 as it enters the canister 12. This helps to ensure that fluid entering the canister 12 does not engage the walls and keeps the flapper door 44 clean. The torsion spring 52 can also be selected with a spring force that allows the flapper door 44 to gap under vacuum when the canister 12 is being emptied. With the door slightly opened by vacuum, air will flow over the flapper door 44 to help clean the flapper door 44. A cleaner can be sprayed onto the flapper door 44 to further facilitate cleaning.

As stated above, the canister 12 collects medical waste, e.g., bodily fluids, tissue etc. from, for example, surgical operations. The fluid and tissue is drawn through the manifold 14 where the tissue is separated by the at least one filter 18 to only allow liquid waste to flow into the canister 12. When the waste collection unit 10 is used with other patients, or if the manifold 14 is full of solid and/or semi-solid material, the manifold 14 is changed. When the manifold 14 is removed, the flapper door 44 closes to prevent fluid within the canister 12 from exiting the canister 12 and to reduce or eliminate any odors from escaping from the canister 12.

When the canister 12 is full it has to be emptied and cleaned. In one embodiment of the invention, the integrated system includes an on-board unit, which mates with a docking station that provides water and cleaner. The on-board unit sprays cleaner and water within the canister 12 to clean the canister 12. In the disclosed embodiment, the flapper door 44 is angled with respect to the inlet port 26 and a vertical axis A of the canister 12 and is recessed into the valve body 38 when the flapper door 44 is closed. More specifically, the base 54 of the valve body 38 has an upper surface 68 and a lower surface 70 oriented at an acute angle relative to the upper surface 68 with the valve body 38 defining a recess in the lower surface 70. The flapper door 44 mates with the recess when in the closed position such that the flapper door 44 is oriented at the acute angle relative to the vertical axis A of the canister 12 to deflect cleaner entering the canister 12 away from the inlet port 26. In this way, the cleaner and water are prevented from exiting the canister 12 through the inlet port 26. The recess also functions as a seal to prevent the water or cleaner from passing around the flapper door 44.

Still referring to FIGS. 8 and 9, a vent 72 is provided in the valve body 38 to vent fluid within the canister 12 of the waste collection unit 10 and prevent pressure buildup within the canister 12. The vent 72 is a port that extends through the valve body 38 from the throat 40. The vent 72 port provides operative communication between the inlet port 26 and the canister 12 when the manifold 14 is removed and the flapper door 44 is in the closed position. As discussed above, the waste collection unit 10 can have an on-board unit for cleaning. This system sprays cleaner and water into the canister 12 to clean the canister 12. The vent 72 allows fluid within the canister 12 to vent to a discharge port (not shown) in the canister 12 and be discharged from the unit to a drain in the event of a system failure, such as for example if an offload pump (not shown) fails. In this way, pressure cannot build within the canister 12 of the waste collection unit 10. The vent 72 is also located so that fluid cannot easily exit the canister 12 through the inlet port 26 either during transportation or offloading. In particular, the vent 72 is horizontally oriented relative to the vertical axis A of the canister 12 and is only slightly spaced from a wall 74 of the cap 24 of the canister 12 to thereby define a treacherous path for liquid waste to follow to reach the inlet port 26.

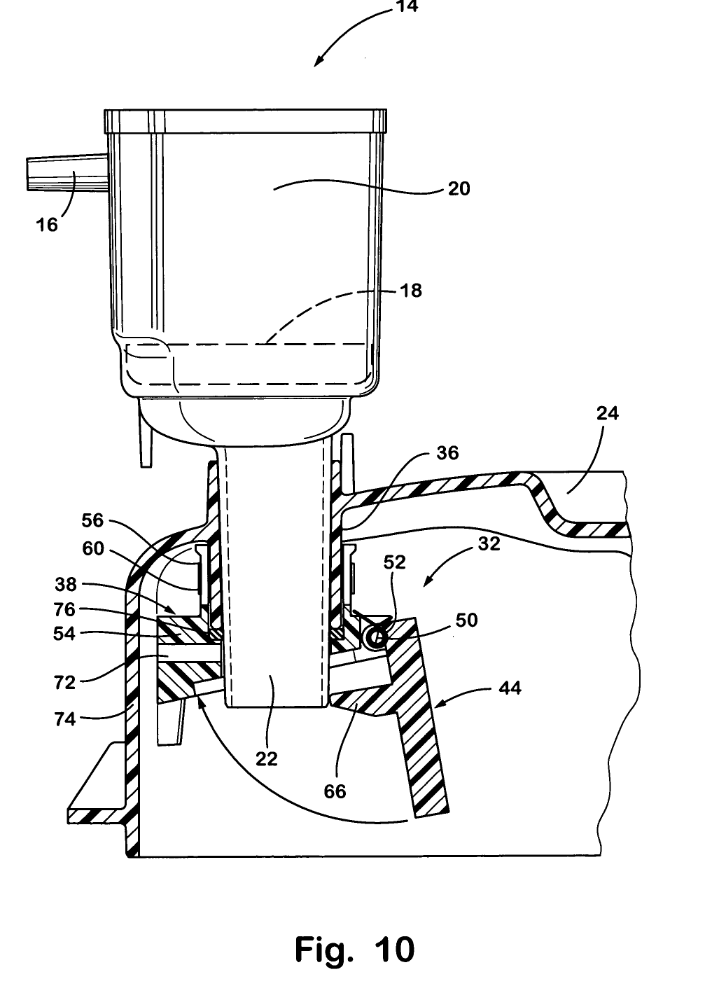

In an alternative embodiment of the manifold interface assembly 30 shown in FIG. 10, the retainer and seal assembly 34 is replaced with a gasket 76 that is captured between the valve body 38 and the inlet member 36.

Obviously, many other modifications and variations of the present intervention are possible in light of the above teachings. The invention may be practiced otherwise than as specifically described within the scope of the appended claims.

1. A waste collection unit for handling medical waste, comprising:
a waste container having an inlet port;
a manifold having at least one filter for filtering the medical waste prior to the medical waste entering said waste container and a stem sized for inserting into said inlet port to couple said manifold to said waste container; and
a manifold interface assembly including a valve assembly having a valve body, a movable member mounted to said valve body and movable between open and closed positions relative to said valve body, and a biasing device for normally biasing said movable member into said closed position to close said inlet port;
wherein said stem has a length sufficiently long to engage said movable member and force said movable member into said open position upon inserting said stem into said inlet port.

2. A waste collection unit as set forth in claim 1 wherein said manifold interface assembly further includes a seal for sealably engaging said stem upon inserting said stem into said inlet port.

3. A waste collection unit as set forth in claim 2 wherein said waste container includes an inlet member defining said inlet port and said manifold interface assembly further includes a retainer mounted to said inlet member about said inlet port with said seal being captured between said retainer and said inlet member.

4. A waste collection unit as set forth in claim 3 wherein said seal is further defined as an o-ring encapsulated in a fluoropolymer.

5. A waste collection unit as set forth in claim 4 wherein said fluoropolymer is further defined as fluorinated ethylene propylene.

6. A waste collection unit as set forth in claim 1 wherein said valve body includes a base and a collar extending upwardly from said base.

7. A waste collection unit as set forth in claim 6 wherein said waste container includes an inlet member defining said inlet port and said collar defines a plurality of slits for providing flexibility to said collar to allow said collar to be clamped about said inlet member.

8. A waste collection unit as set forth in claim 7 wherein said manifold interface assembly includes a clamp disposed about said collar for securing said valve body to said inlet member.

9. A waste collection unit as set forth in claim 1 wherein said movable member is further defined as a flapper door pivotally supported by said valve body to move between said open and closed positions.

10. A waste collection unit as set forth in claim 9 wherein said flapper door includes a first pair of hinge arms and said valve body includes a second pair of hinge arms.

11. A waste collection unit as set forth in claim 10 wherein said manifold interface assembly further includes a pivot pin pivotally coupling said first pair of hinge arms to said second pair of hinge arms to define a hinge.

12. A waste collection unit as set forth in claim 9 wherein said valve body defines a throat extending through said valve body and said biasing device normally biases said flapper door into said closed position to normally close said throat.

13. A waste collection unit as set forth in claim 12 wherein said valve body defines a vent port to prevent pressure buildup within said waste container wherein said vent port extends through said valve body from said throat and said vent port provides operative communication between said inlet port and said waste container when said manifold is removed and said flapper door is in said closed position.

14. A waste collection unit as set forth in claim 1 wherein said movable member is further defined as a flapper door and said biasing device is further defined as a torsion spring acting between said valve body and said flapper door to normally bias said flapper door into said closed position.

15. A waste collection unit as set forth in claim 14 wherein said torsion spring has a spring force that allows said flapper door to gap from said valve body when a vacuum is applied in said waste container thereby drawing air across flapper door to clean said flapper door.

16. A waste collection unit as set forth in claim 1 wherein said valve body includes a base having an upper surface and a lower surface oriented at an acute angle relative to said upper surface with said valve body defining a recess in said lower surface.

17. A waste collection unit as set forth in claim 16 wherein said movable member is further defined as a flapper door pivotally supported by said valve body.

18. A waste collection unit as set forth in claim 17 wherein said flapper door mates with said recess in said closed position such that said flapper door is oriented at an acute angle relative to a vertical axis of said waste container to deflect cleaner entering said waste container away from said inlet port.

19. A waste collection unit for handling medical waste, comprising:
a waste container having an inlet port;
a manifold configured to be insertable into said inlet port to couple said manifold to said waste container, said manifold having at least one filter for filtering the medical waste prior to the medical waste entering said waste container;
a manifold interface assembly including a valve assembly having a valve body, a movable member mounted to said valve body and movable between open and closed positions relative to said valve body, and a biasing device for normally biasing said movable member into said closed position to close said inlet port; and
an actuator for actuating said movable member and forcing said movable member into said open position upon inserting said manifold into said inlet port.
